(19) United States
(12) Patent Application Publication
(10) Pub. No.: US 2019/0374447 A1
(43) Pub. Date: Dec. 12, 2019

(54) DISSOLVABLE OBJECTS AND RELATED METHODS OF MANUFACTURE

(71) Applicant: Robert Nathan Alder, Greenacres, FL (US)

(72) Inventor: Robert Nathan Alder, Greenacres, FL (US)

(21) Appl. No.: 16/550,038

(22) Filed: Aug. 23, 2019

Related U.S. Application Data
(63) Continuation-in-part of application No. 15/959,049, filed on Apr. 20, 2018.
(60) Provisional application No. 62/522,854, filed on Jun. 21, 2017.
(60) Provisional application No. 62/488,481, filed on Apr. 21, 2017.
(60) Provisional application No. 62/722,364, filed on Aug. 24, 2018.

(51) Int. Cl. A61K 8/11; A61K 8/02; A61Q 19/10; A61K 8/04

(52) U.S. Cl. CPC A61K 8/11; A61K 8/0216; A61K 2800/222; A61K 8/046; A61Q 19/10

(57) ABSTRACT

The present disclosure is directed to dissolvable objects in the form of a bar of soap. The dissolvable objects are dissolvable in a solvent, such as water in a bath tub. The dissolvable objects are further configured to release an additive, or a collection of additives, which can be embedded within the bar of soap. The present disclosure is also directed to methods of manufacturing a bar of soap.

### RELATED APPLICATIONS

This application is a continuation-in-part of U.S. application Ser. No. 15/959,049, filed on Apr. 18, 2018 and titled “CUSTOMIZABLE DISSOLVABLE OBJECTS AND RELATED METHODS OF MANUFACTURE”, which claims priority to U.S. Provisional Application No. 62/488,481, filed on Apr. 21, 2017 and titled “CUSTOMIZATION OF DISSOLVABLE OBJECTS,” and to U.S. Provisional Application No. 62/522,854 filed on Jun. 21, 2017 and titled “CUSTOMIZABLE DISSOLVABLE OBJECTS AND RELATED METHODS OF MANUFACTURE,” and claims priority to U.S. Provisional Application No. 62/722,364, filed on Aug. 22, 2019 titled “DISSOLVABLE OBJECTS AND RELATED METHODS OF MANUFACTURE,” each of the foregoing of which is hereby incorporated by reference in its entirety.

### FIELD OF THE INVENTION

The present disclosure relates generally to dissolvable objects that can be put in an agent, liquid, or environment, such as a bath, that causes that object to dissolve and release fragrances and/or oils or other dermatologic or skin-care substances.

### BACKGROUND

Presently, effervescent dissolvable objects (e.g., a bath bomb) include a mixture of reactive ingredients that are combined and formed into a sphere (or other three-dimensional shape). In a formed state, these presently available dissolvable objects are dry and do not react until placed in a liquid which begins to dissolve the sphere and release the contents of the sphere.

The presently available dissolvable objects are distributed premade in a formed state and are not alterable, and cannot be modified or otherwise configured or customized. Presently available dissolvable objects generally cannot be opened, and are not capable of receiving (or of having anything inserted) inside to customize the dissolvable object. For example, the fragrances and/or oils of the dissolvable object cannot be modified or otherwise customized.

Presently available dissolvable objects in the formed (e.g., pre-made, dry) state have a short shelf life of a couple weeks as the fragrances and other ingredients evaporate from the dissolvable object.

Existing manufacturing methods to form dissolvable objects don't allow for any insertion of external items. There would also be a fear of the object collapsing on itself as normal manufacturing is to press two half-sphere molds together firmly to pack and bind the ingredients and contents and to collapse cavities.

The foregoing challenges relating to lack of customizability and longevity of presently available dissolvable objects can be addressed by the disclosed embodiment.

### BRIEF DESCRIPTION OF THE DRAWINGS

FIG. 1A is a cross-sectional view of a dissolvable object, according to one embodiment of the present disclosure, that includes a channel.

FIG. 1B is a cross-sectional view of the dissolvable object of FIG. 1A, with a customization receptacle or capsule inserted into the channel.

FIG. 2 is a cross-sectional view of a dissolvable object, according to another embodiment of the present disclosure, including a channel that is drilled to allow for a customization receptacle or capsule to be inserted inside the object.

FIG. 3 is a cross-sectional view of a dissolvable object, according to another embodiment of the present disclosure, formed in two halves that, when combined, form a cavity and an external item can be inserted in the cavity prior to combining the two halves.

FIG. 4 is a cross-sectional view of a dissolvable object, according to another embodiment of the present disclosure, wrapped in an air-tight outer layer.

FIG. 5A is a cross-sectional view illustrating one step of a method of manufacturing a dissolvable object, according to one embodiment of the present disclosure.

FIG. 5B is a cross-sectional view illustrating another step of a method of manufacturing a dissolvable object, according to the embodiment depicted in FIG. 5A of the present disclosure.

FIG. 5C is a cross-sectional view illustrating yet another step of a method of manufacturing a dissolvable object, according to the embodiment depicted in FIG. 5A of the present disclosure.

FIG. 6A is a cross-sectional view illustrating one step of a method of manufacturing a dissolvable object, according to another embodiment of the present disclosure.

FIG. 6B is a cross-sectional view illustrating another step of a method of manufacturing a dissolvable object, according to the embodiment depicted in FIG. 6A of the present disclosure.

FIG. 7A is a perspective view of a dissolvable object in the form of a soap body, according to an embodiment of the present disclosure.

FIG. 7B is a sectional view of the dissolvable object of FIG. 7A.

FIG. 8 is a sectional view of the water soluble additive capsule of FIG. 7B according to an embodiment of the present disclosure.

FIG. 9 is a partial perspective view of a molding system for manufacturing a dissolvable object in the form of the soap body of FIGS. 7A, 7B, according to an embodiment of the present disclosure.

### DETAILED DESCRIPTION

In the following detailed description, reference is made to the accompanying drawings, which form a part hereof, and in which is shown by way of illustration specific embodiments in which the present disclosure may be practiced. These embodiments are described in sufficient detail to enable those of ordinary skill in the art to practice the disclosure made herein. It should be understood, however, that the detailed description and the specific examples, while indicating examples of embodiments of the disclosure, are given by way of illustration only, and not by way of limitation. From the disclosure, various substitutions, modifications, additions, rearrangements, or combinations thereof within the scope of the disclosure may be made and will become apparent to those of ordinary skill in the art.

In one embodiment of the present disclosure, one or more channels is molded or cut into a dissolvable object so as to allow an external item or liquid to be inserted inside a main object body of the dissolvable object.

In another embodiment of the present disclosure, a cavity can be created within a main object body of a dissolvable object to allow a space for an external item to rest and be trapped or retained until the outer main object body dissolves.

In another embodiment of the present disclosure, an access channel can be formed within a main object body to insert an external object. The access channel can be left open or closed with a plug or door.

In another embodiment of the present disclosure, a liquid or powder is contained within a dissolvable object, and the liquid or powder is released and can mix with other contents of the dissolvable object.

In another embodiment of the present disclosure, a liquid or powder is contained within a dissolvable object and releases or mixes with other contents, including the main object body (i.e. a bath bomb that is wrapped in a dissolvable wrapper/shell/capsule/receptacle).

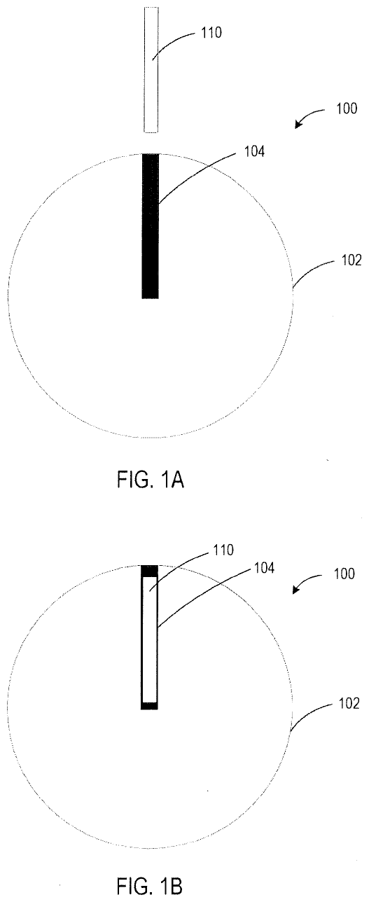

FIG. 1A is a dissolvable object (e.g., a bath enhancing article) 100, according to one embodiment of the present disclosure. The dissolvable object includes an object body 102 and a channel 104 formed in the object body 102. The dissolvable object of FIG. 1A further includes one or more customization capsules 110 (or receptacles, liquids, or other selectable additives) that can be inserted into and received within the channel 104. The channel 104 may be formed in the object body 102 of the dissolvable object 100 during a manufacturing process so that when the object body 102 dries and/or cures to a formed state the shape of a channel and/or cavity will remain in the object body 102. In other embodiments, the channel 104 is formed after the object body 102 dries and or cures. The channel 104 may pass part or all the way through the object body 102.

The customization capsule 110 may be formed of a dissolvable film, wrapper, shell, or the like, or other material that may similarly dissolve in a dissolving liquid or solvent that dissolves the object body 102. The customization capsules 110 may include one or more customization substances or additives in a liquid or powder form, such as fragrances, vitamins, nutrients, antioxidants, stimulants, pheromones, and/or oils or other dermatologic or skin-care substances or substances that can be absorbed by the skin into the body. These customization substances are secured within the capsule retained from evaporation or other dissipation from passage of time. Moreover, the customization capsule(s) 110 or liquid enables a user of the dissolvable object to select a combination of customization substances or additives according to a present preference or desire prior to placement of the dissolvable object 100 into bath water.

FIG. 1B is the dissolvable object 100 of FIG. 1A, with a customization capsule 110 inserted into the channel 104. The customization capsule 110 may be secured within the channel 104 by friction (e.g., a friction fit), a plug, a door, or other securement mechanism. When placed in a liquid (e.g., water) or other solvent able to dissolve the object body 102, the dissolvable object 100 gradually dissolves, initially by dissolution of the object body 102 and eventually including dissolution of the customization capsule 110. The eventual dissolution of the customization capsule 110 releases a desired combination of customization substances into the solution.

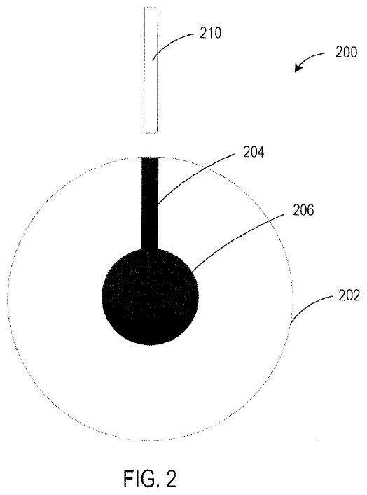

FIG. 2 is a dissolvable object 200, according to another embodiment of the present disclosure. The dissolvable object 200 includes a cavity 204 that may be drilled or otherwise to provide such capabilities to allow for an external object 210 to be inserted inside a cavity 206 within an object body 202 of the dissolvable object 200. The external object 210 may be a customization capsule, one or more capsules of essential oils, toys, flowers, pheromones, vitamins, antioxidants, fragrances, nutrients, stimulants, other chemicals, or other dermatologic or skin-care substances or substances that can be absorbed by the skin into the body. The external object 210 may be inserted inside the hole and may be lodged inside the channel 204, or rest inside the cavity 206 to prevent escape.

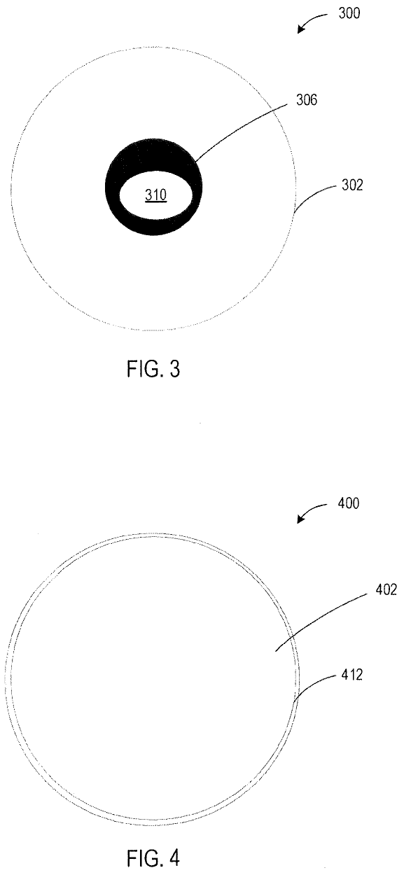

FIG. 3 is a cross-sectional view of a dissolvable object 300, according to another embodiment of the present disclosure. The dissolvable object 300 includes an object body 302 that is formed in two halves that, when combined, form a cavity 306. An external item 310 can be inserted in the cavity 306 prior to combining the two halves of the object body 302. The external item 310 may be a capsule of oil or vitamins etc.

In still another embodiment, the dissolvable object 300 may be simply formed around an external item 310, such as a capsule. The capsule may include oil or vitamins, etc., but the capsule or other protective coating around the oil or vitamins, etc. may prolong a shelf life of the dissolvable object 300, albeit without allowing customization after formation.

FIG. 4 is a cross-sectional view of a dissolvable object 400, according to another embodiment of the present disclosure. The dissolvable object 400 includes a main body 402 and a dissolvable outer layer 412 that encloses (e.g., wraps, seals, covers) the main body 402 to preserve any fragrances that may be included in the main body. Stated differently, a main body 402 (e.g., formed of an effervescent powder) may be sealed in a dissolvable air tight packaging to prolong a shelf life of the main body 402.

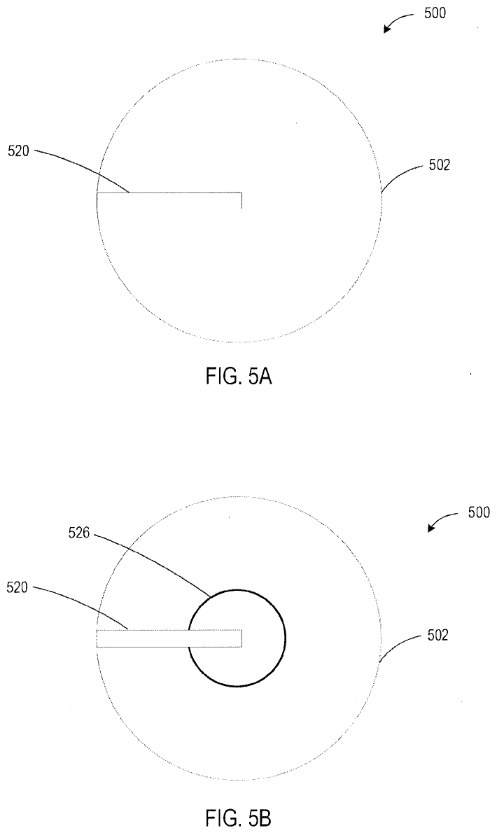

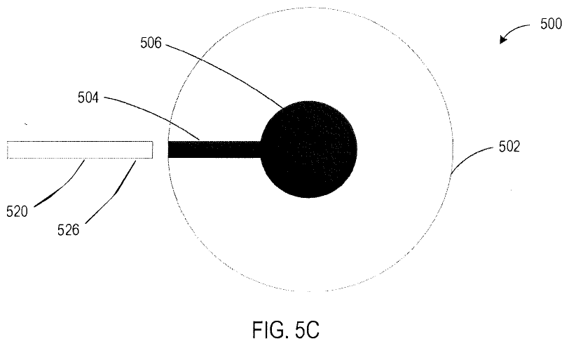

FIG. 5A is a cross-sectional view illustrating one step of a method of manufacturing a dissolvable object 500, according to one embodiment of the present disclosure. The dissolvable object 500 includes a main body 502 that may be penetrated by an external device 520 configured to penetrate into the main body 502 of the dissolvable object 500. For example, the external device 520 may remove a portion of the material comprising the main body 502 of the dissolvable object 500. A drilling action of the external device 520 may remove material of the main body 502 to form a channel 504. In another embodiment, the external device 520 may be forcefully inserted into the main body 502, such as after the manner of taking a core sample, thereby displacing the material of the main body 502.

FIG. 5B is a cross-sectional view illustrating another step of the method of manufacturing a dissolvable object 500 of FIG. 5A. The dissolvable object 500 includes a main body 502 that may have been penetrated by an external device 520 configured to be inserted into the main body 502 of the dissolvable object 500. The external device 520 of FIG. 5B includes a cavity formation tip 526 that may be configured to displace or remove additional material within the main body 502 at a distal end of the external device 520 after the device has been inserted. For example, the cavity formation tip 526 may include an expandable distal end configured to expand to displace additional material within the main body 502 when the expandable distal end is pressurized. In one embodiment, a fluid (i.e., hydraulic forces) may be used to pressurize the expandable distal end of the cavity formation tip 526 thereby causing a portion of the material within the main body 502 to be displaced. Alternatively, in other embodiments, gas (i.e., pneumatic forces) may be used to pressurize the expandable distal end of the cavity formation tip 526 thereby causing a portion of the material within the main body 502 to be displaced.

FIG. 5C is a cross-sectional view illustrating yet another step of the method of manufacturing a dissolvable object 500, of FIG. 5A. The dissolvable object 500 includes a main body 502 that may have been penetrated by an external device 520 configured to be inserted into the main body 502 of the dissolvable object 500 and displace or remove material at an expandable distal end of the external device 520. After the material within the main body 502 has been removed or displaced, the expandable distal end of the external device 520 may be retracted from a cavity 506 formed by the external object 520 (e.g., with the expandable distal end of the cavity formation tip 526) within the main body 502 of the dissolvable object 500. As the external device 520 is also removed, a channel 504 remains formed within the main body 502 of the dissolvable object 500.

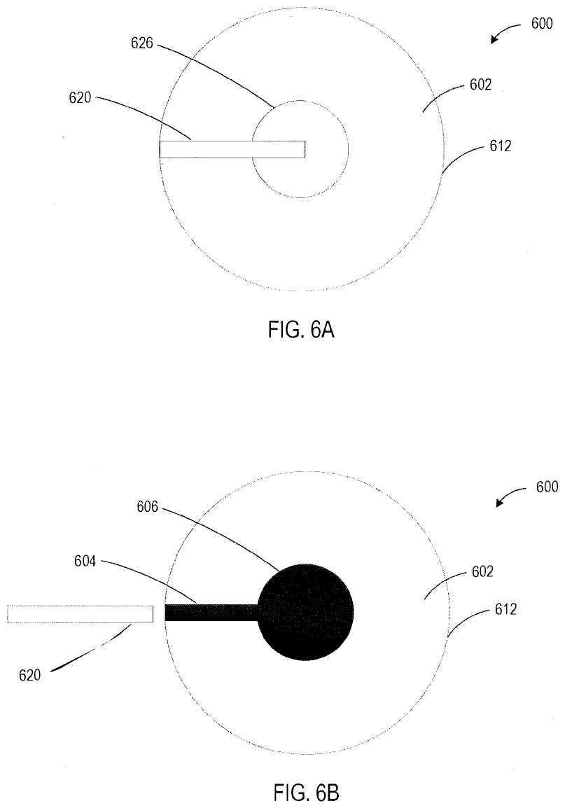

FIG. 6A is a cross-sectional view illustrating one step of a method of manufacturing a dissolvable object 600, according to another embodiment of the present disclosure. The dissolvable object 600 may be formed from a mold 612. The mold 612 may be configured to partially encase an external device 620 within the main body 602 of the dissolvable object 600 within the mold 612. The external device 620, partially encased within the mold 612, includes a cavity formation tip 626 that may be configured to inhibit material placed within the mold 612 from occupying a portion of the volume within the mold 612. When material is placed within the mold 612, the expandable distal end of the external device 620 may be expanded and thereby inhibit material placed within the mold 612 from occupying the entirety of the volume within the mold 612. For example, the cavity formation tip 626 may include an expandable distal end configured to expand to occupy a portion of the volume within the mold 612 when the expandable distal end is pressurized. In one embodiment, a fluid (i.e., hydraulic forces) may be used to expand the expandable distal end of the cavity formation tip 626 thereby occupying a portion of the volume within the mold 612. Alternatively, in other embodiments, gas (i.e., pneumatic forces) may be used to pressurize the expandable distal end of the cavity formation tip 626 thereby occupying a portion of the volume within the mold 612 when the expandable distal end of the cavity formation tip 626 is pressurized.

FIG. 6B is a cross-sectional view illustrating another step of the method of manufacturing a dissolvable object 600, of FIG. 6A. The mold 612, partially encasing an external device 620 having a cavity formation tip 626, may be filled with a material to create a dissolvable object 600. After the material placed within the mold 612 has hardened (e.g., compressed, cured, set, etc.), the expandable distal end of the cavity formation tip 626 may be retracted or rescinded as to allow removal of the external device 620 from the main body 602 of the dissolvable object 600 and the mold 612 and leave a cavity 606 and channel 604 within the mold 612 free of material.

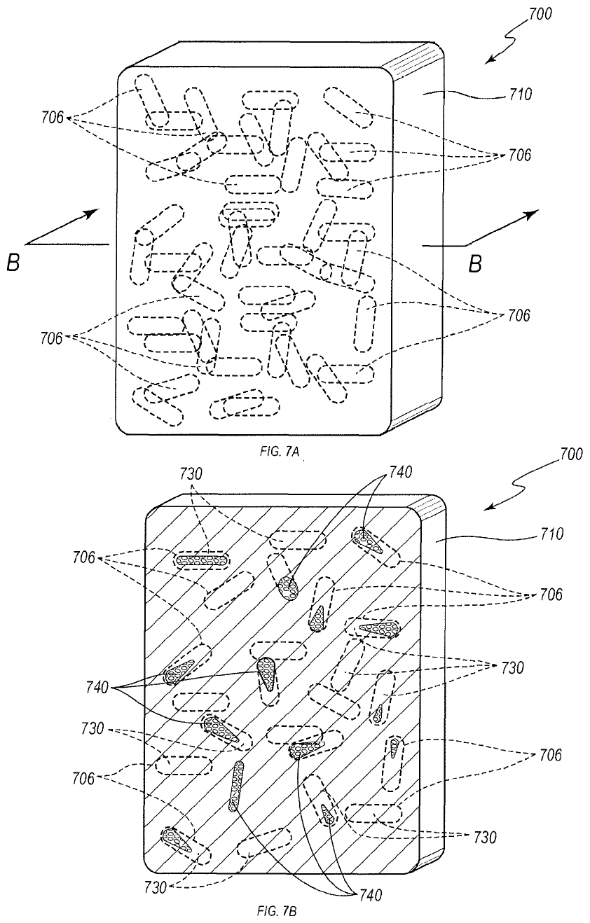

FIG. 7A is a perspective view of a dissolvable object 700 in the form of a soap body 710, according to an embodiment of the present disclosure. The soap body 710, here illustrated as a bar, may take any appropriate form. A plurality of cavities 706 are disposed within the soap body 710. The cavities 706 may be randomly disposed within the soap body 710.

FIG. 7B is a sectional view of the dissolvable object 700 of FIG. 7A. The section angle is shown in FIG. 7A at B. The dissolvable object 700 is in the form a soap body 710. With the soap body 710 shown in section, a number of the cavities 706 are exposed or partially exposed. The cavities 706 may be formed by water soluble additive capsules 730. Each water soluble capsules 730 may be incorporated into the soap body 710 at the time of manufacture, as described below, to form the cavity 706, and, more particularly, to include within the soap body 710 an additive 740. The additive 740 within each capsule 730 may comprise any of an effervescing compound, a skin nutrient (such as, e.g., a protein, a vitamin, etc.) an odorant, or other desirable component. The sectional view of FIG. 7B illustrates some of the cavities 706 exposed or partially exposed, with some of the water soluble additive capsules 730 likewise exposed or partially exposed and cutaway to reveal an additive 740. The additive 740 may be the same in each water soluble additive capsule 730, or may vary among the water soluble additive capsules 730 to create a customized soap body 710.

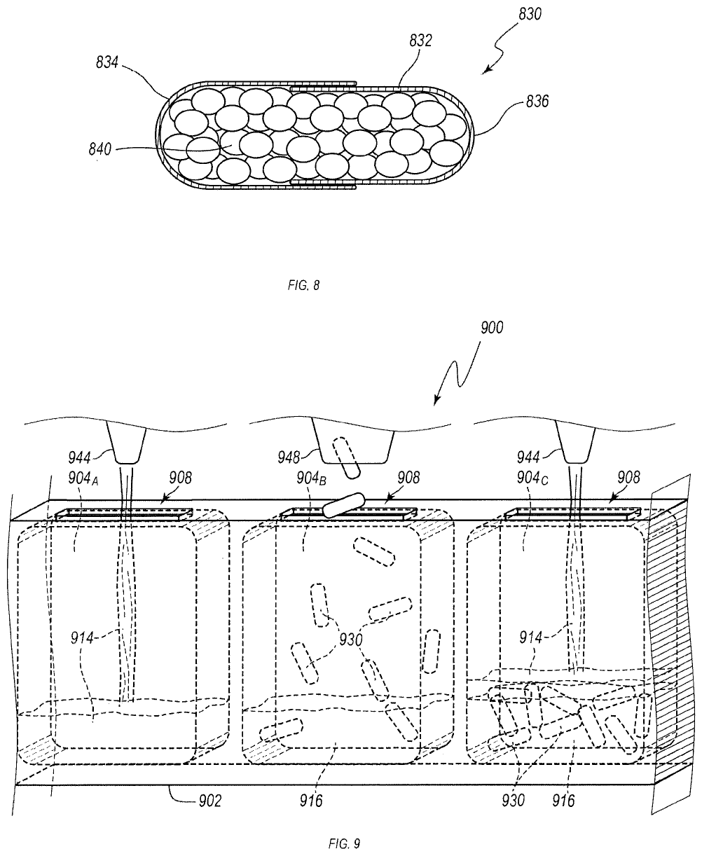

FIG. 8 is a sectional view of the water soluble additive capsule 830 of FIG. 7B according to an embodiment of the present disclosure. The water soluble additive capsule 830 comprises a capsule shell 832. The capsule shell 832 is soluble in water. In one embodiment, the capsule shell 832 may comprise a capsule shell first half 834 and a capsule shell second half 834. The water soluble additive capsule 830 may enclose an additive 840 desirable for customizing the soap body (see 710 in FIGS. 7A, 7B). The additive 840 may be, by way of example without limitation, an effervescing compound, a skin nutrient (such as, e.g., a vitamin, a protein, etc.), an odorant, etc. While a granular representation of the additive 840 is illustrated, this is for convenience only, as the additive 840 may be of any form that does not interact with (dissolve) the water soluble additive capsule 830.

FIG. 9 is a partial perspective view of a molding system 900 for manufacturing a dissolvable object in the form of the soap body of FIGS. 7A, 7B, according to an embodiment of the present disclosure. The molding system 900 comprises a mold body 902 having at least one mold cavity 904. The mold body 902 further comprises a mold aperture 908 for each mold cavity 904. In the embodiment of FIG. 9, the mold body 902 comprises a plurality of mold cavities (collectively, 904_{X}), of which three mold cavities 904_{A}, 904_{B} and 904_{C} are shown.

During manufacture, the material which ultimately forms the soap body (see 710 in FIGS. 7A, 7B) may, at various times, have a liquid, semi-solid, or solid state. In other words, the soap body 710 may be formed by pouring soap in a liquid state into a mold, allowing the soap to cure (or solidify), then removing the soap body 710 from the mold body 902 (or removing the mold body 902 from the soap body 710). Each of the mold cavities 904_{X} in FIG. 9 represents a different step in the manufacture of the soap body 710. At mold cavity 904_{A}, a first discrete quantity of liquid state soap 914 may be introduced to the mold cavity 904_{A} through a liquid state soap nozzle 944. Once the first discrete quantity of liquid state soap 914 has been introduced into the mold cavity 904_{A}, the liquid state soap 914 may be allowed to cure for a discrete period of time so as to attain a semi-solid state. At mold cavity 904_{B}, the soap has cured to at least a semi-solid state 916, and a number of water soluble additive capsules 930 are introduced into the semi-solid soap 916 via a capsule nozzle 948. The semi-solid state soap 916 does not present water in sufficient quantity to dissolve the water soluble capsules 930. Furthermore, the water soluble additive capsules 930 may disburse more or less uniformly throughout the semi-solid state soap 916 such that the water soluble additive capsules 930 are at least most incorporated into the semi-sold state soap 916.

At mold cavity 904_{C}, a second discrete quantity of liquid state soap 914 is introduced into the mold cavity 904_{C} on top of the semi-solid state soap 916. The process of alternatively adding liquid state-soap 914 and, after a discrete curing period for the liquid state soap 914 to attain at a least semi-solid 916, water soluble additive capsules 930 into a mold cavity 904_{X} may be repeated until the mold cavity 904_{X} is filled. Once the soap body 710 achieves a uniform solid state, the soap body 710 may be removed from the mold body 910.

While nozzles 944, 948 are illustrated in FIG. 9, this is for convenience only, and other suitable means of introducing liquid state soap 914 and water soluble additive capsules 930 are also contemplated by the disclosure. Similarly, the illustration of three mold cavities 904_{X} is but one version of a mold body 902, and other mold body 901 configurations are likewise contemplated by the disclosure.

As noted above, each water soluble additive capsule 930 may contain any of an effervescing compound, a skin nutrient, an odorant, etc. A customized soap body 710 may be formed by disposing a plurality of water soluble additive capsules 930 in the soap body 710 wherein the plurality of water soluble additive capsules 930 comprise some water soluble additive capsules 930 containing an effervescing compound, and some water soluble additive capsules 930 contain a skin nutrient, etc.

1. A dissolvable object comprising:
a dissolvable object body; and
a cavity disposed within the dissolvable object body;
wherein the cavity retains an external item until the dissolvable object body dissolves.

2. The dissolvable object of claim 1, wherein the dissolvable object comprises a soap material.

3. The dissolvable object of claim 1, wherein the dissolvable object comprises an effervescent powder.

4. The dissolvable object of claim 1, wherein the external item is a water-soluble capsule.

5. The dissolvable object of claim 4, wherein the water-soluble capsule comprises:
a water-soluble outer shell; and
an additive.

6. The dissolvable object of claim 5, wherein the additive comprises any one or more of an effervescing compound, a skin nutrient, an odorant, a fragrance, a vitamin, an oil, and a dermatologic substance.

7. A dissolvable object comprising:
a soap body; and
a plurality of cavities disposed within the soap body;
wherein each cavity of the plurality of cavities is formed by and contains a water-soluble capsule.

8. The dissolvable object of claim 7, wherein the soap body comprises a soap material.

9. The dissolvable object of claim 7, wherein the soluble capsule comprises:
a water-soluble outer shell; and
an additive.

10. The dissolvable object of claim 9, wherein the additive comprises any one or more of an effervescing compound, a skin nutrient, an odorant, a fragrance, a vitamin, an oil, and a dermatologic substance.

11. A method of manufacturing a, comprising:
pouring molten dissolvable material into a dissolvable object mold;
embedding in the molten dissolvable material a plurality of soluble capsules, each capsule enclosing an additive to be released as the dissolvable object dissolves and soluble material of the capsule is exposed and dissolves;
allowing the dissolvable object to cool and/or cure; and
removing the dissolvable object from the mold.

12. The method of claim 11, wherein each soluble capsule encloses any of an effervescing compound, a skin nutrient, an odorant, a fragrance, a vitamin, an oil, and a dermatologic substance

13. The method of claim 11, wherein the molten dissolvable material is poured in discrete portions at discrete intervals into a mold, wherein the discrete interval between each pouring permits the molten dissolvable material to at least partially cool and/or cure to a temperature tolerable to avoid damaging the soluble capsules as they are embedded therein.

14. The method of claim 13, wherein the plurality of the soluble capsules is added to the partially cooled and/or cured molten dissolvable material between each pouring of the molten dissolvable material.
